(19) United States
(12) Patent Application Publication
(10) Pub. No.: US 2016/0113592 A1
(43) Pub. Date: Apr. 28, 2016

(54) SYSTEM AND METHOD FOR ACQUISITION SETUP AND ANATOMY LANDMARKING OF MAGNETIC RESONANCE IMAGING SYSTEMS

(71) Applicant: General Electric Company, Schenectady, NY (US)

(72) Inventors: Sundar Murugappan, San Ramon, CA (US); Adrian Jeremy Knowles, Brookfield, WI (US); Alexander Kaber Carroll, San Ramon, CA (US)

(21) Appl. No.: 14/526,055

(22) Filed: Oct. 28, 2014

(51) Int. Cl. A61B 5/00; A61B 5/055; A61B 19/00

(52) U.S. Cl. CPC A61B 5/7285; A61B 5/743; A61B 5/7485; A61B 19/54; A61B 5/0555; A61B 19/44; A61B 2019/5437

(57) ABSTRACT

A system and method for acquisition setup and anatomy landmarking for MRI systems are described. A gesture sensing input device generates user motion data. A gesture application identifies a gesture based on the user motion data. A display device displays patient setup information in response to the gesture. An acquisition application generates a setup for an MRI system for a patient based on the gesture and the user motion data.

### TECHNICAL FIELD

The subject matter disclosed herein generally relates to setup or configuration of Magnetic Resonance Imaging (MRI) systems. Specifically, the present disclosure addresses a system and method for hands-free acquisition setup, and visual guidance for anatomy landmarking.

### BACKGROUND

An acquisition workflow of a setup of an MRI procedure starts from the time a patient arrives at the MRI procedure location and is prepared for the scanning procedure. The acquisition workflow may pertain to the interactions between the MRI technologist and the patient. An “in-Room Operating Console” (iROC) enables the MRI technologist to complete pre-examination steps while standing next to the patient. For instance, the iROC enables the MRI technologist to input patient information, cardiac-gating, landmarking and coil setup. Landmarking is the process of identifying scanning areas of the body of the patient. The display of the iROC is typically positioned on top of the bore of the MRI system with two input consoles on either side of the bore. This layout is impractical because the MRI technologist has to constantly shift focus between the input console and the display, moving their head up and down where the distance between the two devices can be significantly large. In addition, the user interface (UI) for entering and editing patient information may be time consuming and not user-friendly, leaving the MRI technologist to finish entering the patient information at an operator console outside the magnet room.

### BRIEF DESCRIPTION OF THE INVENTION

In one embodiment, a system and method for acquisition setup, configuration, and anatomy landmarking for MRI systems are described. A gesture sensing input device generates user motion data. A gesture application identifies a gesture based on the user motion data. A display device displays patient setup information in response to the gesture. An acquisition application generates a configuration for an MRI system for a patient based on the gesture and the user motion data.

### BRIEF DESCRIPTION OF THE DRAWINGS

Some embodiments are illustrated by way of example and not limitation in the figures of the accompanying drawings.

FIG. 1 is a block diagram illustrating a top view of an example of a system for acquisition setup and landmarking of an MRI system, according to some example embodiments.

FIG. 2 is a block diagram illustrating another view of an example of a system for acquisition setup and landmarking of an MRI system, according to some example embodiments.

FIG. 3 is a block diagram illustrating an example embodiment of an MRI setup system.

FIG. 4 is a block diagram illustrating an example embodiment of a patient selection module.

FIG. 5 is a diagram illustrating an example embodiment of a physical characteristics estimation module.

FIG. 6 is a diagram illustrating an example of a landmarking application.

FIG. 7 is a flowchart illustrating an example operation of patient selection of the MRI setup system.

FIG. 8 is a flowchart illustrating an example operation of patient weight estimation of the MRI setup system.

FIG. 9 is a flowchart illustrating an example operation of landmarking of the MRI setup system.

FIG. 10 is a block diagram illustrating components of a machine, according to some example embodiments, able to read instructions from a machine-readable medium and perform any one or more of the methodologies discussed herein.

### DETAILED DESCRIPTION

Example methods and systems are directed to acquisition setup and anatomy landmarking for MRI systems. Examples merely typify possible variations. Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments. It will be evident to one skilled in the art, however, that the present subject matter may be practiced without these specific details.

A system and method for acquisition setup and anatomy landmarking for MRI systems are described. In one example embodiment, an MRI setup system has a gesture sensing input device, a gesture application, a display device, and an acquisition application. The gesture sensing input device generates user motion data. The gesture application identifies a gesture based on the user motion data. The display device displays patient setup information in response to the gesture. The acquisition application generates a setup for an MRI system for a patient based on the gesture and the user motion data.

In one example embodiment, the MRI setup system comprises a landmarking display device that generates a display of a visual guide on the body of the patient. The visual guide identifies scanning boundaries on the body of the patient to the MRI system.

In another example embodiment, the gesture sensing input device generates patient motion data for the patient and technician motion data for the technician setting up the MRI system. The user motion data includes patient motion data and technician motion data.

In another example embodiment, the acquisition application comprises a patient selection module, a physical characteristics estimation module, and a landmarking application. The patient selection module identifies and selects the patient from a list of patients in response to the gesture. The physical characteristics estimation module determines a height and a weight of the patient using the gesture sensing input device, and adjusts a position of a table of the MRI system in response to the height and weight of the patient. The height and weight information may also be used to estimate a Specific Absorption Rate (SAR). The landmarking application identifies, using the gesture sensing input device, a portion of a body of the patient to be scanned using the MRI system.

In another example embodiment, the patient selection module comprises a patient status module and a patient selection gesture module. The patient status module identifies a list of patients associated with the MRI system, the patient from the list of patients, a status of the patient, a procedure associated with the patient, and a body position of the patient using the gesture sensing input device. The patient selection gesture module selects the patient from the list of patients based on the displayed patient setup information using the gesture sensing input device.

In another example embodiment, the physical characteristics estimation module comprises a height computation module and a weight computation module. The height computation module estimates a height of the patient based on patient motion data generated by the gesture sensing input device. The weight computation module calculates a weight of the patient based on the height of the patient and the patient motion data generated by the gesture sensing input device.

In another example embodiment, the landmarking application comprises a speech recognition module, a landmark sensing module, and a landmark visual guide. The speech recognition module receives audio commands from a technician of the MRI system. The landmark sensing module identifies the portion of the body of the patient to be scanned in response to a combination of gesture and audio commands from the technician of the MRI system. The landmark visual guide generates a visual indicator projected on the portion of the body of the patient. The visual indicator identifies scanning boundaries for MRI system.

In another example embodiment, the MRI setup system comprises an audio sensing input device that generates technician command data. The gesture application is responsive to the technician command data.

In another example embodiment, the display device is disposed parallel to a table of the MRI system. The display device generates a display for the visual guide identifying where the patient is to sit on a table of the MRI system, which side the patient is to lay on the table, and in which direction the patient is to be positioned relative to a bore of the MRI system. The display device may include a projection device that generates a display on a screen disposed parallel to the table of the MRI system.

In another example embodiment, a non-transitory machine-readable storage device may store a set of instructions that, when executed by at least one processor, cause the at least one processor to perform the method operations discussed within the present disclosure.

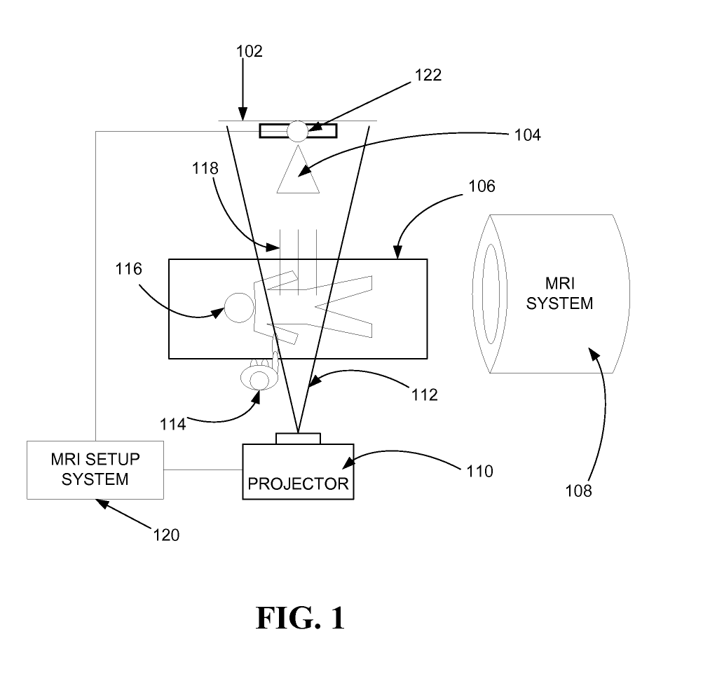

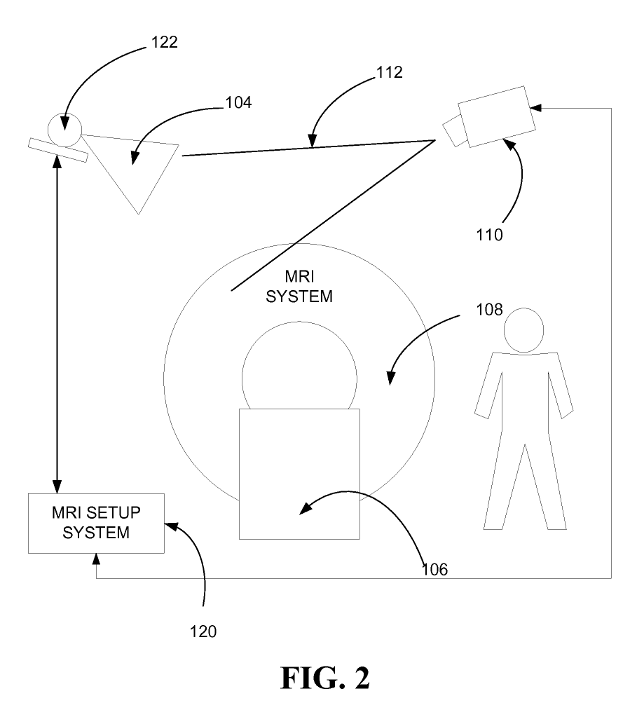

FIG. 1 is a block diagram illustrating a top view of an example of a system for acquisition setup and landmarking of an MRI system, according to some example embodiments. FIG. 2 is a block diagram illustrating a side view of the system for acquisition setup and landmarking of the MRI system. An operator 114 (e.g., an MRI technologist or MRI technician), prepares an MRI system 108 with the help of an MRI setup system 120 to set up an MRI scanning procedure for a patient 116 laying on a table 106. The MRI setup system 120 focuses on the workflow pertaining to the operator 114, the patient 116, and the interactions between the operator 114 and the patient 116, and provides an alternative to the ‘in-Room Operating Console (iROC)’ where the operator 114 completes pre-examination steps while standing next to the patient 116. The MRI system 108 may include a bore with strong magnetic fields and radio waves to form images of the body. The strong magnetic field is to be positioned around an area to be imaged.

The MRI setup system 120 may be connected to a projector 110, a gesture sensing input device 122 disposed against a wall 102 opposite to the projector 110. The gesture sensing input device 122 may include optical sensors such as infrared and depth sensors configured to capture stereoscopic images of the operator 114 and the patient 116. The optical sensors may have a field of view 104 that includes a portion of the body of the patient 116 and the operator 114. The gesture sensing input device 122 can generate stereoscopic images to determine the distance of the operator 114 to the gesture sensing input device 122 and recognize hand gestures of the operator 114. Common gesture sensing input devices may capture a depth image of a scene to determine whether objects in the scene correspond to a human body shape of the operator 114. A skeletal model may be generated based on the depth images and the movement of the operator 114. The MRI setup system enables the operator to operate hands-free using gestures sensed by the gesture sensing input device 122 and perform a pre-examination workflow of the corresponding patient 116 while standing next to the patient without having to look away from the patient 116. The operator 114 may navigate through a workflow process by performing predefined gestures corresponding to predefined movements of the skeletal model of the operator 114. For example, a movement of the right arm from right to left may correspond to a forward command through the workflow process. One of ordinary skill in the art will recognize that gestures may be defined to correspond to different types of commands. The gestures may be further enhanced and complemented with voice input command from the operator 114.

The projector 110 may include any type of projection imaging device that generates a projection 112 of an image or a moving image onto a screen on the wall 102. In one example embodiment, the projector 110 generates a visual feedback onto the wall 102 for the operator 114. In another example embodiment, the projector 110 may generate another visual feedback onto a portion of the body of the patient 116. For example, the projector 110 may project visual markings 118 identifying landmarking positions for the scanning of the patient 116. Landmarking positions identify regions of the body of the patient 116 where the MRI system 108 is to perform the scanning. The visual markings 118 may be represented by parallel lines: two lines indicating boundaries of the scanning region, one line indicating a center or middle portion of the scanning boundary region.

In another example embodiment, the projector 106 may be used to project visual feedback information for the operator 114. Another projector (not shown) may be used to project visual feedback information on a portion of the body of the patient 116. The visual feedback information may include, for example, the visual marking 118 (e.g., lines or visual indicators) that identify portions of the body to be scanned. The operator 114 performs gestures to adjust and redefine the scanning region defined by the visual marking 118. For example, the operator 114 may perform a predefined gesture (e.g., moving hands outward) associated with expanding the scanning region. The operator 114 may position his arms and hands above the corresponding portion of the body of the patient 116 to be scanned. The operator 114 may further adjust the size of the region using voice command and/or gestures.

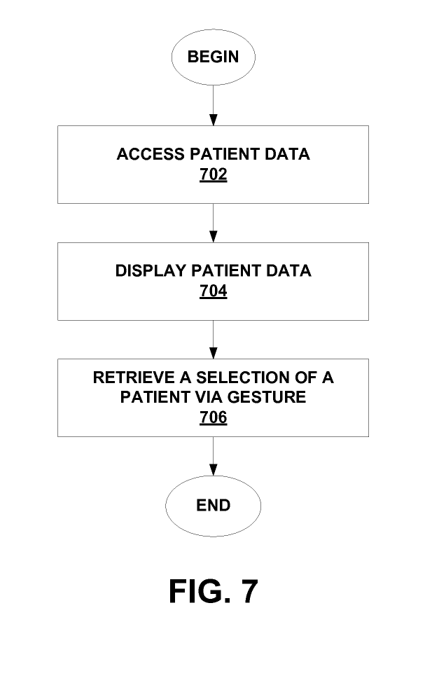

Example operations of the MRI setup system 120 are described in more detail with respect to FIGS. 7, 8, and 9 below. Some benefits of the MRI setup system 120 include hands-free interaction within the magnet room where the MRI system 108 is located. Hands-free operation reduces contamination in the room and related problems. The MRI setup system 120 further provides for tracking and identification of the operator 114 in the room, visual estimation of height and weight of the patient 116, automatic lowering of the table 106, speech recognition input, visual feedback guidance for the patient 116 to lie on the table 106 (e.g., on which side (prone/supine) and in which direction (head-first/leg-first)). The MRI setup system 120 further provides live visual guidance for new and inexperienced operators or technologists to navigate through the workflows (e.g., acquisition setup and landmarking). The MRI setup system 120 provides real-time information and status of multiple events in the corresponding workflow (e.g., where the patient 116 is right now, what kind of scan is needed), and automatic identification of patient 116's anatomy profile.

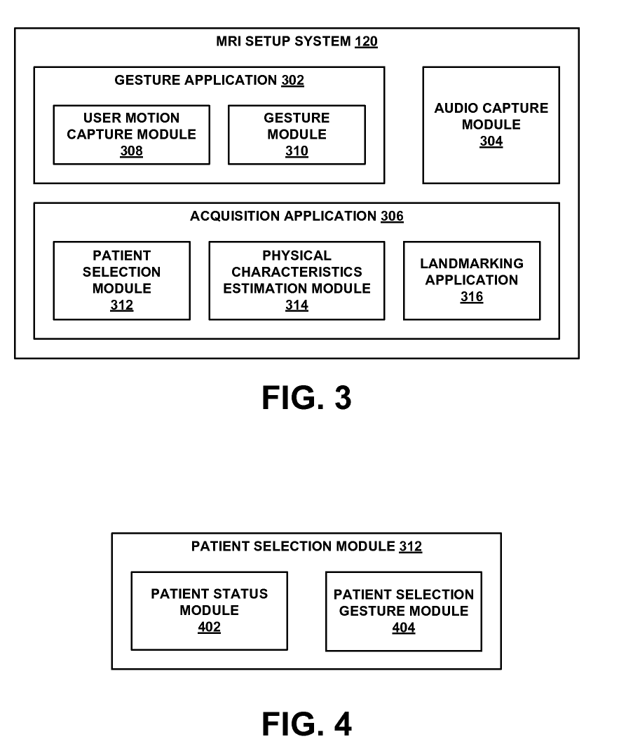

FIG. 3 is a block diagram illustrating an example embodiment of the MRI setup system 120. The technical effect of the MRI setup system 120 is to enable a technician or operator to efficiently generate a configuration of an MRI system for a patient. The MRI setup system 120 may include a computer including a gesture application 302, an audio capture module 304, and an acquisition application 306. The gesture application 302 may identify a gesture based on the user motion data generated by the gesture sensing input device 122. In an example embodiment, the gesture application 302 includes a user motion capture module 308 and a gesture module 310. The user motion capture module 308 generates user motion data that include patient motion data for the patient and technician motion data for the technician setting up the MRI system 108. For example, the patient motion data may include a skeletal model of the body of the patient on the table 106 or standing in a predefined area in the room. The patient motion data may be based on body motion of the patient and is used to generate a skeletal model with corresponding motion. The technician motion data may include a skeletal model of the body of the technician in a predefined area in the room (area next to and adjacent to the table 106). The technician motion data may be based on body motion of the technician and is used to generate a skeletal model with corresponding motion. In one embodiment, a single gesture sensing input device 122 may be used to capture both the patient motion data and technician motion data. In another example embodiment, a gesture sensing input device may be dedicated to generating patient motion data. Another gesture sensing input device may be dedicated to generating technician motion data.

The gesture module 310 interprets the motion data of the operator 114 and the patient 116 to identify a corresponding gesture. For example, the motion data may indicate left to right arm motions of the operator 114. Such motion data may be associated with a gesture indicating a predefined command. The gesture module 310 may be programmed to associate commands for the MRI setup system 120. In one embodiment, the gesture module 310 may discriminate gestures from the operator 114 and the patient 116. For example, the gesture module 310 may ignore gestures from the patient 116 when the operator 114 is performing a gesture.

The audio capture module 304 may generate commands based on audio input captured from the operator 114. For example, the operator 114 may issue voice commands to the MRI setup system 120. The audio capture module 304 may include a voice recognition system to identify word commands from the operator 114 and retrieve commands or functions associated with the identified word commands.

The acquisition application 306 may include a patient selection module 312, a physical characteristics estimation module 314, and a landmarking application 316. The patient selection module 312 identifies a list of patients associated with the MRI system 108 and their corresponding status. For example, the list of patients identifies patients that are to be present on the day of the scanning. The patient selection module 312 further enables the operator 114 to select and identify the patient 116 from the list of patients. A status of the patient 116 is updated accordingly. For example, the status may include no-show, waiting in reception area, and present. The patient selection module 312 further identifies procedures associated with the patient 116, and a body scanning region of the patient 116. Components of the patient selection module 312 are further described with respect to FIG. 4 below.

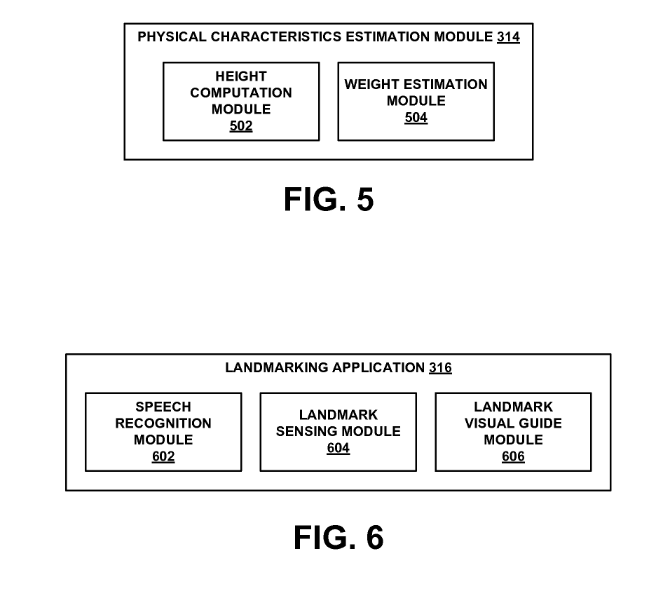

The physical characteristics estimation module 314 determines a height and a weight of the patient 116 using the gesture sensing input device 122. For example, the gesture sensing input device 122 may be used to compute an estimated height and weight of the patient 116 based on the skeletal model of the patient 116. The physical characteristics estimation module 314 may further adjust a position of the table 106 of the MRI system 108 in response to the estimated height and weight of the patient 116. For example, the physical characteristics estimation module 314 may lower a height of the table 106 to accommodate a relatively short patient 116 or raise the height of the table 106 to accommodate a relatively tall patient 116. Components of the physical characteristics estimation module 314 are further described with respect to FIG. 5 below.

The landmarking application 316 may identify, using the gesture sensing input device 122, a portion of the body of the patient 116 to be scanned using the MRI system 108. For example, the landmarking application 316 enables the operator 114 to identify the scanning portion of the body of the patient 116 using voice and gesture commands without the operator 114 having to access a keyboard or touching the table 106. Components of the landmarking application 316 are further described with respect to FIG. 6 below.

FIG. 4 is a block diagram illustrating an example embodiment of the patient selection module 312. The patient selection module 312 may include a patient status module 402 and a patient selection gesture module 404. The patient status module 402 accesses a list of patients associated with the MRI procedure on a particular day. The patient status module 402 enables the operator 114 to select and view information related to the patients. For example, the information may include name, birthday, gender, address, MRI scanning region (e.g., left knee). The patient selection gesture module 404 enables the operator 114 to navigate through the list of patients using predefined gestures (e.g., waving right hand from right to left to scroll through the list). Other gestures may be predefined to enable the operator 114 to select a patient's name from the list of patients and to update their corresponding status (e.g., waiting in lobby, no show, late). In another embodiment, the patient selection gesture module 404 may use speech recognition to enhance the accuracy of the gestures. For example, the operator 114 may say “select” to choose an identified patient from the list of patients. The patient selection gesture module 404 may access a database of tables to retrieve functions or commands corresponding to predefined gestures from the operator 114. In another embodiment, the database may also include a table of commands and corresponding gestures from the patient. For example, the patient selection gesture module 404 may generate a skeletal model of the patient 116 and determine that the patient 116 is laying still on the table. Music or calm scenery may be displayed in the room to further relax the patient 116 in response to determining that the patient 116 is still on the table. For example, pictures of palm trees on an island may be projected or displayed onto the walls/ceiling of the MRI room to create an immersive, relaxing environment. In another embodiment, the patient selection gesture module 404 includes a facial recognition algorithm that identifies the patient 116 from the list of patients. The patient selection gesture module 404 retrieves information (e.g., patient chart, patient profile including music and scenery preference) relevant to the identified patient 116 based on the facial features of the patient 116. The patient selection gesture module 404 may trigger the facial recognition process when the patient 116 enters the MRI room and faces the gesture sensing input device 122 or is within a field of view 104 of the gesture sensing input device 122.

FIG. 5 is a block diagram illustrating an example embodiment of the physical characteristics estimation module 314. The physical characteristics estimation module 314 may include a height computation module 502 and a weight estimation module 504. As previously described, the gesture sensing input device 122 may be used to generate a skeletal model of the patient 116. The patient 116 may be asked to stand at a predefined area in the room or lie down on the table 106. The gesture sensing input device 122 generates the skeletal model of the patient 116 based on the patient 116 being present in the predefined area. The height computation module 502 generates an estimation of the height of the patient 116 based on the skeletal model. The weight estimation module 504 generates an estimation of the weight of the patient 116 based on the skeletal model of the patient 116. In another embodiment, the height and weight of the patient 116 may be determined using a built-in scale on the table 106 and identifying measurement markers on the table 106. The measurement markers may identify distances on the table 106 (e.g., a mark every inch). The gesture sensing input device 122 may include an optical device configured to identify the position of the body of the patient 116 relative to the measurement markers on the table 106. The physical characteristics estimation module 314 may adjust a position of the table 106 based on the height and weight of the patient 116.

FIG. 6 is a block diagram illustrating an example of the landmarking application 316. The landmarking application 316 may include a speech recognition module 602, a landmark sensing module 604, and a landmark visual guide module 606. As previously described, the landmarking application 316 identifies a portion of the body of the patient 116 to be scanned using the MRI system 108 by using the gesture sensing input device 122. The speech recognition module 602 may be configured to receive audio commands from the operator 114. For example, the operator 114 may say “set landmark” to identify and submit the region of the body to be scanned. The speech recognition module 602 may be optionally used to enhance the accuracy of detecting gestures from the operator 114. The landmark sensing module 604 detects gestures of the operator 114 to identify the portion of the body of the patient 116 to be scanned. For example, the operator 114 may extend his hands and arms over portions of the body of the patient 116 to identify boundaries of the region to be scanned. The landmark sensing module 604 may identify the position of the hands and arms of the operator 114 relative to the body of the patient 116 and determine the region to be scanned based on the position of the hands and arms of the operator 114. For example, the left hand of the operator 114 may be positioned above the pelvis of the patient 116 and the right hand of the operator 114 may be positioned above the heart of the patient 116. The boundaries defined by the hands of the operator 114 would thus include the abdomen area of the patient 116. The landmark sensing module 604 is able to set the scanning area as the abdomen area. As such, the operator 114 stands next to the patient 116 and faces the patient 116 and the wall 102. The gesture sensing input device 122 detects gestures from the operator 114 relative to the detected body of the patient 116 to identify the scanning boundaries.

The landmark visual guide module 606 may generate a visual indicator to indicate a sitting area on the table 106 and an orientation in which the patient 116 is to lie on the table 106. The visual indicators may be generated with another projector aimed at the table 106. In another embodiment, the visual indicators may be displayed on the surface of the table 106 via other means (e.g., embedded display or lights). For example, the visual indicators may include a shaded sitting area projected on the table 106 or displayed on the table 106. The shaded sitting area provides a cue for the patient 116 to sit on the table 106 at the shaded sitting area. The location of the shaded sitting area may be based on the information from the chart of the patient 116, and the estimated height and weight of the patient 116. In another example, the visual indicators include an arrow projected on the table 106 or displayed on a surface of the table 106 to indicate an orientation or direction for the patient 116 to lie on the table (e.g., head first or feet first towards the MRI system 108). In another example, the landmark visual guide module 606 may cause a visual outline of the body of the patient 116 to be displayed or projected on the surface of the table 106 with the body direction based on the information in the chart of the patient 116. In another example, embodiment the landmark visual guide module 606 may cause a visual avatar of the body of the patient 116 to be displayed or projected on the surface of the table 106 with the body orientation (e.g., prone/supine) based on the information in the chart of the patient 116. The visual avatar may include a capture image of the patient with the gesture sensing input device 122. For example, an image of the patient 116 is shown lying in a supine position on the table 106.

FIG. 7 is a flowchart illustrating an example operation of patient selection of the MRI setup system 120. At operation 702, the MRI setup system 120 accesses patient data stored in a repository or database system. Patient data information includes names of patients to be scanned on a particular day, age, gender, height, weight, birthdate, and physician ordered MRI scanning region. At operation 704, the patient data is displayed on the wall 102 opposite to the location of the standing operator 114. At operation 706, the MRI setup system 120 retrieves a selection of a patient via detected gestures using the gesture sensing input device 122.

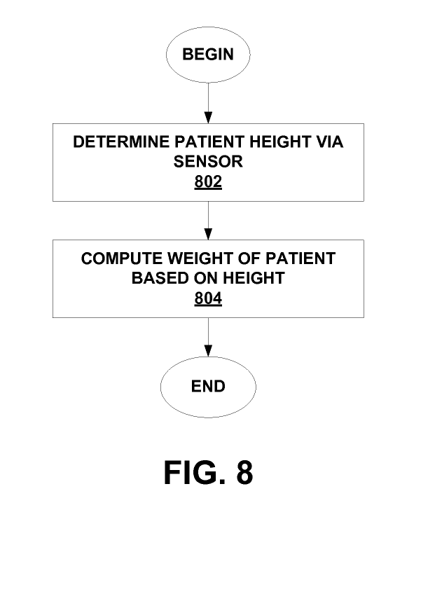

FIG. 8 is a flowchart illustrating an example operation of patient weight estimation of the MRI setup system 120. At operation 802, the MRI setup system 120 determines and estimates the height of the patient 116 using the gesture sensing input device 122. At operation 804, the MRI setup system 120 computes an estimated weight of the patient 116 based on the estimated height of the patient.

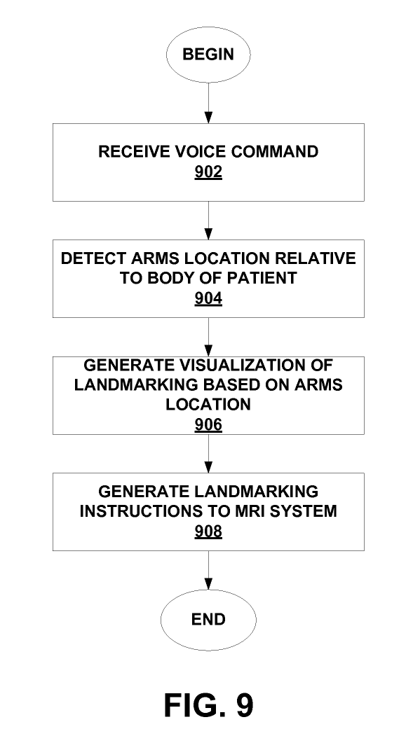

FIG. 9 is a flowchart illustrating an example operation of landmarking of the MRI setup system 120. At operation 902, the MRI setup system 120 receives a voice command from the operator 114 to initiate the landmarking process. In one example embodiment, operation 902 may be implemented using the speech recognition module 602 of the landmarking application 316. At operation 904, the MRI setup system 120 uses the gesture sensing input device 122 to detect the location of operator 114's arms relative to the body of the patient 116. In one example embodiment, operation 904 may be implemented using the landmark sensing module 604 of the landmarking application 316. At operation 906, the MRI setup system 120 generates a visualization of landmarking based on the detected operator 114 arms location. In one example embodiment, operation 906 may be implemented using the landmark visual guide module 606 of the landmarking application 316. At operation 908, the MRI setup system 120 generates landmarking instructions to the MRI system 108.

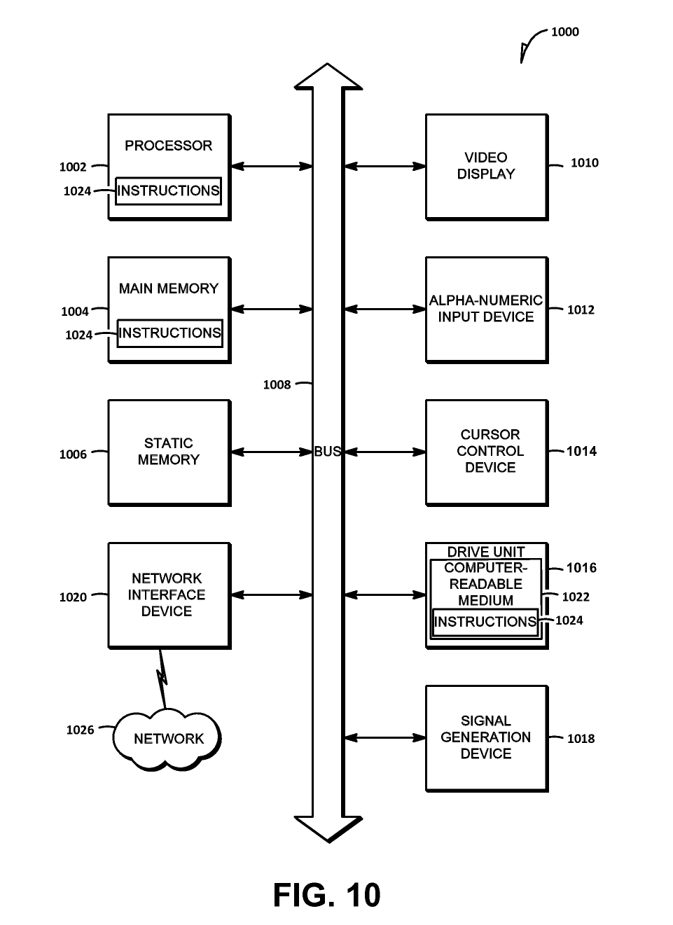

Any of the machines, databases, or devices shown in FIG. 3 may be implemented in a general-purpose computer modified (e.g., configured or programmed) by software to be a special-purpose computer to perform one or more of the functions described herein for that machine, database, or device. For example, a computer system able to implement any one or more of the methodologies described herein is discussed below with respect to FIG. 10. As used herein, a “database” is a data storage resource and may store data structured as a text file, a table, a spreadsheet, a relational database (e.g., an object-relational database), a triple store, a hierarchical data store, or any suitable combination thereof. Moreover, any two or more of the machines, databases, or devices illustrated in FIG. 1 may be combined into a single machine, and the functions described herein for any single machine, database, or device may be subdivided among multiple machines, databases, or devices.

The MRI setup system 120 may communicate over a computer network that may be any network that enables communication between or among machines (e.g., MRI system 108), databases, and devices (projector 110). Accordingly, the network may be a wired network, a wireless network (e.g., a mobile or cellular network), or any suitable combination thereof. The network may include one or more portions that constitute a private network, a public network (e.g., the Internet), or any suitable combination thereof.

### Modules, Components and Logic

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client, or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods described herein may be at least partially processor-implemented. For example, at least some of the operations of a method may be performed by one or more processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network and via one or more appropriate interfaces (e.g., APIs).

### Electronic Apparatus and System

Example embodiments may be implemented in digital electronic circuitry, or in computer hardware, firmware, software, or in combinations of them. Example embodiments may be implemented using a computer program product, e.g., a computer program tangibly embodied in an information carrier, e.g., in a machine-readable medium for execution by, or to control the operation of, data processing apparatus, e.g., a programmable processor, a computer, or multiple computers.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network (e.g., network 1026 of FIG. 10).

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

A computing system can include clients and servers. A client and server are generally remote from each other and typically interact through a communication network. The relationship of client and server arises by virtue of computer programs running on the respective computers and having a client-server relationship to each other. In embodiments deploying a programmable computing system, it will be appreciated that both hardware and software architectures merit consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

### Example Machine Architecture and Machine-Readable Medium

FIG. 10 is a block diagram of a machine in the example form of a computer system 1000 within which instructions 1024 for causing the machine to perform any one or more of the methodologies discussed herein may be executed. In alternative embodiments, the machine operates as a standalone device or may be connected (e.g., networked) to other machines. In a networked deployment, the machine may operate in the capacity of a server or a client machine in a server-client network environment, or as a peer machine in a peer-to-peer (or distributed) network environment. The machine may be a personal computer (PC), a tablet PC, a set-top box (STB), a personal digital assistant (PDA), a cellular telephone, a web appliance, a network router, switch or bridge, or any machine capable of executing instructions 1024 (sequential or otherwise) that specify actions to be taken by that machine. Further, while only a single machine is illustrated, the term “machine” shall also be taken to include any collection of machines that individually or jointly execute a set (or multiple sets) of instructions 1024 to perform any one or more of the methodologies discussed herein.

The example computer system 1000 includes a processor 1002 (e.g., a central processing unit (CPU), a graphics processing unit (GPU) or both), a main memory 1004 and a static memory 1006, which communicate with each other via a bus 1008. The computer system 1000 may further include a video display unit 1010 (e.g., a liquid crystal display (LCD) or a cathode ray tube (CRT)). The computer system 1000 also includes an alphanumeric input device 1012 (e.g., a keyboard), a user interface (UI) navigation (or cursor control) device 1014 (e.g., a mouse), a disk drive unit 1016, a signal generation device 1018 (e.g., a speaker) and a network interface device 1020.

### Machine-Readable Medium

The disk drive unit 1016 includes a computer-readable medium 1022 on which is stored one or more sets of data structures and instructions 1024 (e.g., software) embodying or utilized by any one or more of the methodologies or functions described herein. The instructions 1024 may also reside, completely or at least partially, within the main memory 1004 and/or within the processor 1002 during execution thereof by the computer system 1000, the main memory 1004 and the processor 1002 also constituting machine-readable media 1022. The instructions 1024 may also reside, completely or at least partially, within the static memory 1006 (not shown).

While the machine-readable medium 1022 is shown in an example embodiment to be a single medium, the term “machine-readable medium” may include a single medium or multiple media (e.g., a centralized or distributed database, and/or associated caches and servers) that store the one or more instructions 1024 or data structures. The term “machine-readable medium” shall also be taken to include any tangible medium that is capable of storing, encoding or carrying instructions 1024 for execution by the machine and that cause the machine to perform any one or more of the methodologies of the present embodiments, or that is capable of storing, encoding or carrying data structures utilized by or associated with such instructions 1024. The term “machine-readable medium” shall accordingly be taken to include, but not be limited to, solid-state memories, and optical and magnetic media. Specific examples of machine-readable media 1022 include non-volatile memory including, by way of example, semiconductor memory devices (e.g., erasable programmable read-only memory (EPROM), electrically erasable programmable read-only memory (EEPROM), and flash memory devices); magnetic disks such as internal hard disks and removable disks; magneto-optical disks; and compact disc-read-only memory (CD-ROM) and digital versatile disc (or digital video disc) read-only memory (DVD-ROM) disks.

### Transmission Medium

The instructions 1024 may further be transmitted or received over a communications network 1026 using a transmission medium. The instructions 1024 may be transmitted using the network interface device 1020 and any one of a number of well-known transfer protocols (e.g., HTTP). Examples of communication networks include a LAN, a WAN, the Internet, mobile telephone networks, POTS networks, and wireless data networks (e.g., WiFi and WiMax networks). The term “transmission medium” shall be taken to include any intangible medium capable of storing, encoding, or carrying instructions 1024 for execution by the machine, and includes digital or analog communications signals or other intangible media to facilitate communication of such software.

Although an embodiment has been described with reference to specific example embodiments, it will be evident that various modifications and changes may be made to these embodiments without departing from the broader spirit and scope of the present disclosure. Accordingly, the specification and drawings are to be regarded in an illustrative rather than a restrictive sense. The accompanying drawings that form a part hereof, show by way of illustration, and not of limitation, specific embodiments in which the subject matter may be practiced. The embodiments illustrated are described in sufficient detail to enable those skilled in the art to practice the teachings disclosed herein. Other embodiments may be utilized and derived therefrom, such that structural and logical substitutions and changes may be made without departing from the scope of this disclosure. This Detailed Description, therefore, is not to be taken in a limiting sense, and the scope of various embodiments is defined only by the appended claims, along with the full range of equivalents to which such claims are entitled.

Such embodiments of the inventive subject matter may be referred to herein, individually and/or collectively, by the term “invention” merely for convenience and without intending to voluntarily limit the scope of this application to any single invention or inventive concept if more than one is in fact disclosed. Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separate embodiment.

What is claimed is:

1. A system to configure a Magnetic Resonance Imaging (MRI) system, the system comprising:
a gesture sensing input device configured to generate user motion data;
a gesture application, implemented using a hardware processor of a machine, configured to identify a gesture based on the user motion data;
a display device configured to display patient setup information in response to the gesture; and
an acquisition application configured to configure the MRI system for a patient based on the gesture and the user motion data.

2. The system of claim 1, further comprising:
a landmarking display device configured to display a visual guide on a body of the patient, the visual guide identifying scanning boundaries on the body of the patient to the MRI system.

3. The system of claim 1, wherein the gesture sensing input device is configured to generate patient motion data for the patient and technician motion data for the technician setting up the MRI system, the user motion data comprising the patient motion data and the technician motion data.

4. The system of claim 1, wherein the acquisition application comprises:
a patient selection module configured to identify and select the patient from a plurality of patients in response to the gesture;
a physical characteristics estimation module configured to determine a height and a weight of the patient using the gesture sensing input device, and to adjust a position of a table of the MRI system in response to the height and weight of the patient; and
a landmarking application configured to identify, using the gesture sensing input device, a portion of a body of the patient to be scanned using the MRI system.

5. The system of claim 4, wherein the patient selection module comprises:
a patient status module configured to identify a list of patients associated with the MRI system, the patient from the list of patients, a status of the patient, a procedure associated with the patient, and a body position of the patient using the gesture sensing input device; and
a patient selection gesture module configured to select the patient from the list of patients based on the displayed patient setup information using the gesture sensing input device.

6. The system of claim 4, wherein the physical characteristics estimation module comprises:
a height computation module configured to estimate a height of the patient based on patient motion data generated by the gesture sensing input device; and
a weight computation module configured to calculate a weight of the patient based on the height of the patient and the patient motion data generated by the gesture sensing input device.

7. The system of claim 4, wherein the landmarking application comprises:
a speech recognition module configured to receive audio commands from a technician of the MRI system;
a landmark sensing module configured to identify the portion of the body of the patient to be scanned in response to the gesture and audio commands from the technician of the MRI system; and
a landmark visual guide configured to generate a visual indicator projected on the portion of the body of the patient, the visual indicator identifying scanning boundaries for the MRI system.

8. The system of claim 1, further comprising:
an audio sensing input device configured to generate technician command data, the gesture application responsive to the technician command data.

9. The system of claim 1, wherein the display device is disposed parallel to a table of the MRI system, the display device further configured to display a visual guide identifying where the patient is to sit on the table of the MRI system, which side of the patient's body is to lie on the table, and which direction the patient is to be positioned relative to a bore of the MRI system.

10. The system of claim 1, wherein the display device comprises a projection device, the projection device configured to generate a display on a screen disposed parallel to a table of the MRI system.

11. A method for configuring a Magnetic Resonance Imaging (MRI) system, the method comprising:
generating user motion data using a gesture sensing input device;
identifying, using a hardware processor of a machine, a gesture based on the user motion data;
displaying patient setup information in response to the gesture;
generating a configuration for the MRI system for a patient based on the gesture and the user motion data; and
providing the configuration to the MRI system.

12. The method of claim 11, further comprising:
generating a display of a visual guide on a body of the patient, the visual guide identifying scanning boundaries on the body of the patient to the MRI system.

13. The method of claim 11, further comprising:
generating patient motion data for the patient and technician motion data for the technician setting up the MRI system, the user motion data comprising the patient motion data and the technician motion data.

14. The method of claim 11, further comprising:
identifying and selecting the patient from a plurality of patients in response to the gesture;
determining a height and a weight of the patient using the gesture sensing input device;
adjusting a position of a table of the MRI system in response to the height and weight of the patient; and
identifying, using the gesture sensing input device, a portion of a body of the patient to be scanned using the MRI system.

15. The method of claim 14, further comprising:
identifying a list of patients associated with the MRI system, the patient from the list of patients, a status of the patient, a procedure associated with the patient, and a body position of the patient using the gesture sensing input device; and
selecting the patient from the list of patients based on the displayed patient setup information using the gesture sensing input device.

16. The method of claim 14, further comprising:
estimating a height of the patient based on patient motion data generated by the gesture sensing input device; and
calculating a weight of the patient based on the height of the patient and the patient motion data generated by the gesture sensing input device.

17. The method of claim 14, further comprising:
receiving audio commands from a technician of the MRI system;
identifying the portion of the body of the patient to be scanned in response to the gesture and audio commands from the technician of the MRI system; and
generating a visual indicator projected on the portion of the body of the patient, the visual indicator identifying scanning boundaries for MRI system.

18. The method of claim 11, further comprising:
displaying a visual guide on a display device disposed parallel to a table of the MRI system, the visual guide identifying where the patient is to sit on a table of the MRI system, which side of the patient's body is to lay on the table, and which direction the patient is to be positioned relative to a bore of the MRI system.

19. The method of claim 11, further comprising:
using a projection device to generate a display on a screen disposed parallel to a table of the MRI system.

20. A non-transitory machine-readable medium comprising instructions that, when executed by one or more processors of a machine, cause the machine to perform operations comprising:
generating user motion data using a gesture sensing input device;
identifying a gesture based on the user motion data;
displaying patient setup information in response to the gesture; and
providing a setup for an MRI system for a patient based on the gesture and the user motion data.
